(19) United States
(12) Patent Application Publication
(10) Pub. No.: US 2010/0043096 A1
(43) Pub. Date: Feb. 18, 2010

(54) The Method for Enhancement of Photosynthesis and Biomass of Plant by Plastid Transformation of Malate Dehydrogenase

(76) Inventors: Jang Ryol Liu, Taejeon-si (KR); Hwa-Jee Chung, Taejeon-si (KR); Sung Ran Min, Taejeon-si (KR); Won Joong Jeong, Taejeon-si (KR); Hyun Tae Kim, Daegu (KR); Ju Young Park, Taejeon-si (KR); Jung He Hur, Taejeon-si (KR)

Correspondence Address: GREENLEE WINNER AND SULLIVAN P C, 4875 PEARL EAST CIRCLE, SUITE 200, BOULDER, CO, 80301, US

(21) Appl. No.: 12/447,901

(22) PCT Filed: Nov. 2, 2007
(86) PCT No.: PCT/KR07/05529

§ 371 Date: Apr. 29, 2009

(30) Foreign Application Priority Data
Nov. 7, 2006 (KR) 10-2006-0109550

(51) Int. Cl. A01H 5/00; C12N 15/82; A01G 1/00

(52) U.S. Cl. 800/278; 800/298; 800/317.3; 800/320.2; 800/320; 800/313; 800/317.2; 800/312; 800/306; 800/322; 800/314; 800/317.4; 800/317; 800/308; 800/307; 800/309; 800/323; 800/323.1; 800/323.2; 800/323.3; 47/58.1 LS

(57) ABSTRACT

The present invention relates to a method for enhancement of photosynthesis and biomass of a plant by plastid transformation with MDH gene, more precisely a method for enhancement of photosynthesis and biomass of C3 plant by plastid transformation system with MDH gene. The MDH plastid transgenic plant prepared by the method of the present invention exhibits not only increased photosynthesis efficiency but also increased growth rate, leaf area, stem diameter and biomass of the plant, compared with the control plant. Therefore, the plastid transformed C3 plant prepared by C4 type gene introduction can be effectively used for enhancing photosynthesis and biomass of the plant.

### TECHNICAL FIELD

The present invention relates to a method for enhancement of photosynthesis and biomass of plant by plastid transformation with MDH gene, more precisely a method for enhancement of photosynthesis and biomass of C3 plant by plastid transformation system with MDH gene.

### BACKGROUND ART

According to environmental disruption and population increase, it has been a very important issue to improve plant productivity. In parallel with the method for increasing the production of crops by the conventional breeding techniques, attempts to increase the production of crops and biomass using molecular breeding technique have been tried. Particularly, in the field of agriculture and forestry, there has been an attempt to introduce a recombinant nucleic acid molecule prepared by manipulation of a gene related with photosynthesis into a plant to express the molecule therein. This attempt can be made not only in a specific kind of a plant but also in various kinds of plants.

Most of terrestrial higher plants including major agricultural crops such as rice, wheat, barley, soybean and potato assimilate CO₂ in the air through C3 photosynthesis pathway (Calvin cycle). Rubisco (ribulose 1,5-bisphosphate carboxylase/oxygenase), the first enzyme of CO₂ fixation, reacts not only with CO₂ but also with O₂ as well, leading to photorespiration consuming the assimilated carbon (Matsuoka et al., Annu Rev Plant Physiol Plant Mol Biol 52, 297-314, 2001). In the meantime, C4 plants such as corn, sorghum, and sugar cane have an additional C4 photosynthesis pathway increasing CO₂ concentration near Rubisco, which inhibits photorespiration, so that photosynthesis efficiency is much higher than in C3 plants (von Caemmerer and Furbank, Photosynth Res 77, 191-207, 2003; Sage, New Phytologist 161, 341-370, 2004).

Major enzymes involved in C4 photosynthesis pathway are PEP carboxylase, NADP-dependent malate dehydrogenase, NADP-dependent malic enzyme and pyruvate phosphate dikinase. In plants, NADP-dependent malate dehydrogenase (NADP-MDH; EC 1.1.1.82) is a very important enzyme for photosynthesis and exists only in chloroplasts (Sheen, Auun Rev Plant Physiol Plant Mol Biol 50, 187-217, 1999). NADP-dependent MDH converts oxaloacetate into malate in chloroplasts, suggesting that it is involved in photosynthesis (carbon dioxide assimilation) along with NADP-dependent malic enzyme and phosphoenolpyuvate carboxykinase (PCK) and at the same time regulates the release of malate from chloroplasts to cytoplasm (von Caemmerer and Furbank, Photosynth Res 77, 191-207, 2003).

Recently, studies have been focused on enhancement of photosynthesis efficiency in C4 or C3 plants by manipulating genes of the major enzymes involved in C4 plant photosynthesis (Bailey et al., J Exp Bot 51 Spec No, 339-346, 2000; Matsuoka et al., Annu Rev Plant Physiol Plant Mol Biol 52, 297-314, 2001; Hausler et al., J Exp Bot 53, 591-607, 2002; Jeanneau et al., J Exp Bot 53, 1837-1845, 2002; Leegood, J Exp Bot, 53, 581-590, 2002; Rademacher et al., Plant J 32, 25-39, 2002; Miyao, J Exp Bot 54, 179-189, 2003; Chen et al., Planta 219, 440-449. 2004; Izui et al., Annu Rev Plant Biol 55, 69-84, 2004).

Plastid transformation has advantages over nucleus transformation, which is able to increase foreign gene expression dramatically and is a pro-environmental technique by which foreign gene does not exist in pollen, so that this plastid transformation has been a main subject in the field of plant biotechnology. Plastid can be divided into chloroplast involved in photosynthesis, amyloplast involved in amyloid storage, luekoplast not containing pigments, and chromoplast involved in color of flowers and fruits. A plant cell has approximately 200 plastids, and a plastid has 100 genomes and accordingly has 10,000-50,000 copies of genes, while a plant nucleus has generally 1-2 copies of genomes. Thus, the copy number of genome in plastid and the copy number of genome in nucleus are independently maintained. Therefore, if a foreign gene is introduced into plastid as a form of episome, which means plastid is transformed with a foreign gene, a target protein will be mass-produced efficiently, which is incomparable with when nucleus is transformed.

Most reports concerning plastid transformation are made in tobacco and some succeeded the transformation in Arabidopsis thaliana, potato and tomato. However, the transformation efficiency is very low in those plants except tobacco. And even in the transformation of tobacco chloroplast, the promoter/terminator system used for a vector for transformation is originated from tobacco, so that the first homologous recombination induced during the generation of a transformant is followed by another recombination once again in those homologous sequences between terminator region and the tobacco chloroplast genome, resulting in a plant having an abnormal chloroplast genome (Staub J M, Maliga P, Proc. Natl. Acad. Sci. 91, 7468-7472, 1994; Svab Z, Maliga P, Proc. Natl. Acad. Sci. 90, 913-917, 1993) According to the conventional method using the typical vector for plastid transformation, the chances of the secondary recombination that might produce abnormal plastid transformant are approximately 50%. In these cases, successful introduction of a foreign gene and stable expression thereof cannot be guaranteed (Korean Patent Application No. 2006-12477).

The present inventors transformed C3 plant with Corynebacterium glutamicum MDH gene by using plastid transformation system. And the inventors completed this invention by confirming that photosynthesis and biomass of plant were enhanced by plastid transformation.

### DISCLOSURE

### Technical Problem

It is an object of the present invention to provide a transgenic plant with enhanced photosynthesis and biomass, a method for preparing the transgenic plant and a method for enhancing photosynthesis and biomass.

### Technical Solution

To achieve the above object, the present invention provides a transgenic plant generated by inserting MDH (malate dehydrogenase) gene into plastid genome of C3 plant.

The present invention also provides a method for preparing the transgenic plant with MDH gene in its plastid genome, which comprises the following steps: 1) constructing a vector for MDH plastid transformation by inserting MDH (malate dehydrogenase) gene sequence which is active in plastid into a vector for plastid transformation; 2) Preparing a transformant by introducing the vector for MDH plastid transformation into C3 plant or C3 plant culture cell; 3) culturing the transformant of step 2); and 4) re-differentiating the cultured transformant of step 3) by tissue culture.

The present invention further provides a method for enhancing photosynthesis and biomass, which includes the step of cultivating the transgenic plant in outdoors or greenhouse under high luminosity condition.

### ADVANTAGEOUS EFFECT

The transgenic plant with enhanced photosynthesis or biomass generated by MDH plastid transformation of the present invention exhibits remarkably increased oxygen generation, CO₂ absorption, growth rate, leaf area, stem diameter and biomass, compared with control plants, so that it can be effectively used for enhancing photosynthesis and biomass of a plant by providing effective plastid transformed C3 plant.

### DESCRIPTION OF DRAWINGS

The application of the preferred embodiments of the present invention is best understood with reference to the accompanying drawings, wherein:

FIG. 1 is a schematic diagram of a recombinant expression vector for plastid transformation containing rice clp promoter and E. coli rrnB1/B2 terminator:

(a): trnI-trnA region of tobacco plastid genome where a foreign gene is inserted;

(b): CtVG04, the recombinant expression vector for plastid transformation using tobacco rrn promoter and psbA terminator;

(c): pTIA, the basic vector for the construction of the vector for plastid transformation containing tobacco trnI-trnA;

(d): pRclPADGHT, the recombinant vector containing rice clp promoter, aadA gene, gfp gene and rrnB1/B2 terminator; and

(e): pRclPGAH, the recombinant vector in which MCS (multicloning site) is inserted in between rice clp promoter and gfp gene and aadA gene and rrnB1/B2 terminator are linked stepwise.

FIG. 2 is a diagram showing the map of expression vectors for MDH plastid transformation.

FIG. 3 is a diagram showing the results of PCR confirming the presence of MDH in the plastid transgenic plant.

FIG. 4 is a diagram showing the results of Southern blotting confirming that MDH exists in plastids of the T₀ and T₁ generation plastid transgenic plants in the form of homoplasmy.

FIG. 5 is a diagram showing the results of Northern blotting illustrating the expression levels of MDH in plastids of the T₀ and T₁ generation plastid transgenic plants.

FIG. 6 is a graph showing the enzyme activity of MDH in plastid of the T₁ generation plastid transgenic plant.

FIG. 7 is a graph showing the light-dependent CO₂ absorption rate in T₁ generation plastid transgenic plant.

FIG. 8 is a graph showing the Fv/Fm values obtained from the greenhouse cultivation according to the different light conditions (high luminosity, medium luminosity and low luminosity).

FIG. 9 is a diagram showing the phenotype of the period of floral axis development of the T₁ generation plastid transgenic plant cultivated in greenhouse.

FIG. 10 is a graph showing the growth rate of the T₁ generation plastid transgenic plant cultivated in greenhouse from the cultivation to the period of floral axis development.

FIG. 11 is a graph showing the leaf area of the T₁ generation plastid transgenic plant cultivated in greenhouse, measured in the period of floral axis development.

FIG. 12 is a graph showing the stem diameter of the T₁ generation plastid transgenic plant cultivated in greenhouse, measured in the period of floral axis development.

FIG. 13 is a graph showing the live weight and dry weight of biomass (leaves and stems) of the T₁ generation plastid transgenic plant cultivated in greenhouse, measured in the period of floral axis development.

### BEST MODE

Hereinafter, the present invention is described in detail.

The present inventors constructed MDH expression vector by inserting Corynebacterium glytamicum originated MDH gene into the expression vector for plastid transformation (see FIG. 2). Then, seeds of wild type tobacco plant (Nicotiana tabacum, Samsun) were germinated. Leaves of the young plant were separated and explanted in MS medium for further use in plastid transformation. Re-differentiation was repeated in selection medium to induce resistant young shoot to grow a transgenic plant. DNA was extracted from the cultivated plant, followed by PCR to confirm MDH gene introduction (see FIG. 3). Southern blotting was performed with the cultivated T₀ and T₁ generation transformants to investigate whether MDH gene was successfully delivered to the next generation as homoplasmy (see FIG. 4). Northern blotting was also performed with the cultivated T₀ and T₁ generation transformants to confirm the transcription of a target gene was normally induced in plastid genome of the T₀ and T₁ generation transformants (see FIG. 5). After confirming the successful transformation of a plant with MDH gene, MDH enzyme activity was measured. As a result, MDH enzyme activity in the MDH transgenic plant was significantly increased, compared with the control plant (see FIG. 6). CO₂ absorption by photosynthesis was also investigated in the transgenic plant. As a result, CO₂ absorption rate of the transgenic plant was approximately 1.4 fold increased, compared with the control plant (see FIG. 7). Chlorophyll fluorescence is generally released from photosystem 2 (PSII) So, Fv/Fm (the ratio of fluorescence variation to maximum chlorophyll fluorescence) of the dark adapted leaf indicates the maximum value of quantum yield of photochemical reaction. The present inventors measured photosystem II efficiency according to the increase of luminosity in plastid transgenic plants. As a result, Fv/Fm values of plants growing under medium luminosity and low luminosity, regardless of the plastid transgenic plants or the control plants, were all maintained as 0.8. However, when plants were cultivated under high luminosity, the plastid transgenic plant maintained Fv/Fm value as 0.8 but the control plant reduced Fv/Fm value to 0.42. From the results, it was confirmed that photoinhibition by photosystem 2 was not induced in the MDH transgenic plants even under high luminosity condition (see FIG. 8). The present inventors measured growth rate and biomass of the plastid transgenic plants. As a result, growth rate of the transgenic plants was 40-55% increased, compared with the control plants (see FIGS. 9 and 10). In addition, the MDH transgenic plants were confirmed to have 86% increased leaf area, 35% increased stem diameter, 63% increased live weight and 14% increased dry weight (see FIGS. 11-13).

As explained hereinbefore, the introduction of MDH into C3 plant resulted in the enhancement of photosynthesis and biomass of the plant.

The present invention provides a transgenic plant generated by inserting MDH gene into plastid genome of a target plant.

The MDH gene is preferably C4 plant type NADP-dependent MDH gene, but not always limited thereto. The MDH gene is preferably originated from eukaryotes, but not always limited thereto.

The MDH gene herein is preferably originated from one of Corynebacterium glutamicum, Rhodococcus, Oceanobacillus, Aspergillus, Methanothermus, Chaetomium, Methanopyrus, Bacillus, Methanocaldococcus, Magnaporthe, Phaeosphaeria, Gibberella, Desulfitobacterium, coccidioides, Thermus, Candidatus, Pyrococcus, Solibacter, Aurantimonas, Syntrophus, Enterococcus, Methanosphaera, Anopheles, Entamoeba, Yersinia, Mesorhizobium, Tribolium, Salmonella, Aurantimonas, Amycolatopsis, Escherichia coli, Apis, Burkhoderia, Bordetella, Pseudomonas and Aedes, and more preferably originated from Corynebacterium glutamicum, but not always limited thereto. And any MDH gene originated from other bacteria can be used as long as it has at least 30% homology with the amino acid sequence of Corynebacterium glutamicum originated MDH.

A target plant for the transformation is preferably C3 plant, which is selected from the group consisting of tobacco; cereals including rice; beans including mung bean, kidney bean and pea; starch-storing plants including potato, cassava and sweet potato; oil-storing plants including soybean, rape, sunflower and cotton plant; vegetables including tomato, eggplant, carrot, hot pepper, Chinese cabbage, radish, water melon, cucumber, melon, crown daisy, spinach, cabbage and strawberry; garden plants including chrysanthemum, rose, carnation and petunia; and Arabidopsis, but not always limited thereto, and any C3 plant well-known to those in the art can be used.

The present invention also provides a method for preparing the transformant comprising the following steps: 1) constructing a vector for MDH plastid transformation by inserting MDH (malate dehydrogenase) gene sequence which is active in plastid into a vector for plastid transformation; 2) preparing a transformant by introducing the vector for MDH plastid transformation into C3 plant or C3 plant culture cell; 3) culturing the transformant of step 2); and 4) re-differentiating the cultured transformant of step 3) by tissue culture.

In this method, the MDH gene of step 1) is preferably originated from one of Corynebacterium glutamicum, Rhodococcus, Oceanobacillus, Aspergillus, Methanothermus, Chaetomium, Methanopyrus, Bacillus, Methanocaldococcus, Magnaporthe, Phaeosphaeria, Gibberella, Desulfitobacterium, coccidioides, Thermus, Candidatus, Pyrococcus, Solibacter, Aurantimonas, Syntrophus, Enterococcus, Methanosphaera, Anopheles, Entamoeba, Yersinia, Mesorhizobium, Tribolium, Salmonella, Aurantimonas, Amycolatopsis, Escherichia coli, Apis, Burkhoderia, Bordetella, Pseudomonas and Aedes, and more preferably originated from Corynebacterium glutamicum, but not always limited thereto. And any MDH gene originated from other bacteria can be used as long as it has at least 30% homology with the amino acid sequence of Corynebacterium glutamicum originated MDH.

In this method, the expression vector for plastid transformation of step 1) is the recombinant expression vector containing the construct composed of a plant plastid originated promoter that has low homology with the polynucleotide of the genome of a target plant plastid, rbs sequence, and a terminator that has low homology with the polynucleotide of the genome of a target plant plastid in that order (see FIG. 2). The target plant is preferably C3 plant, which is selected from the group consisting of tobacco; cereals including rice; beans including mung bean, kidney bean and pea; starch-storing plants including potato, cassava and sweet potato; oil-storing plants including soybean, rape, sunflower and cotton plant; vegetables including tomato, eggplant, carrot, hot pepper, Chinese cabbage, radish, water melon, cucumber, melon, crown daisy, spinach, cabbage and strawberry; garden plants including chrysanthemum, rose, carnation and petunia; and Arabidopsis, but not always limited thereto, and any C3 plant well-known to those in the art can be used.

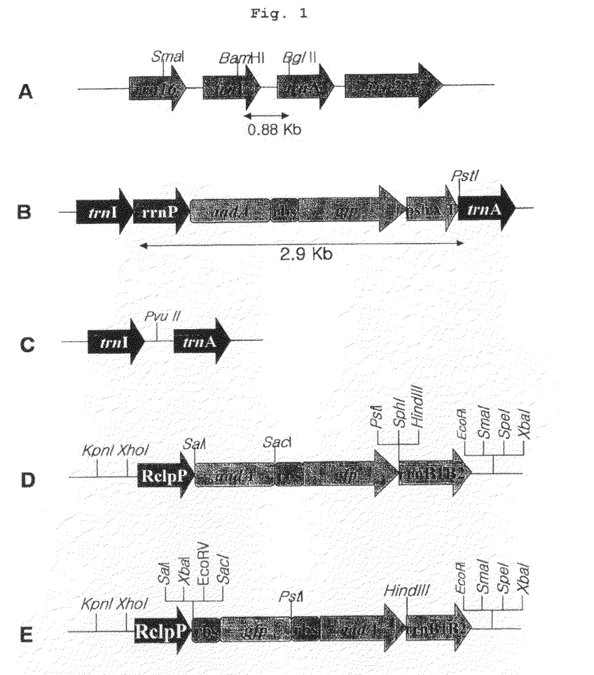

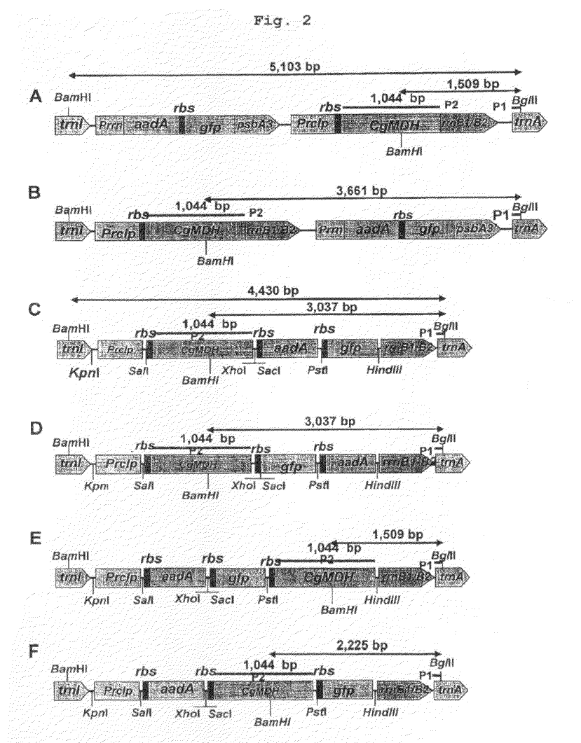

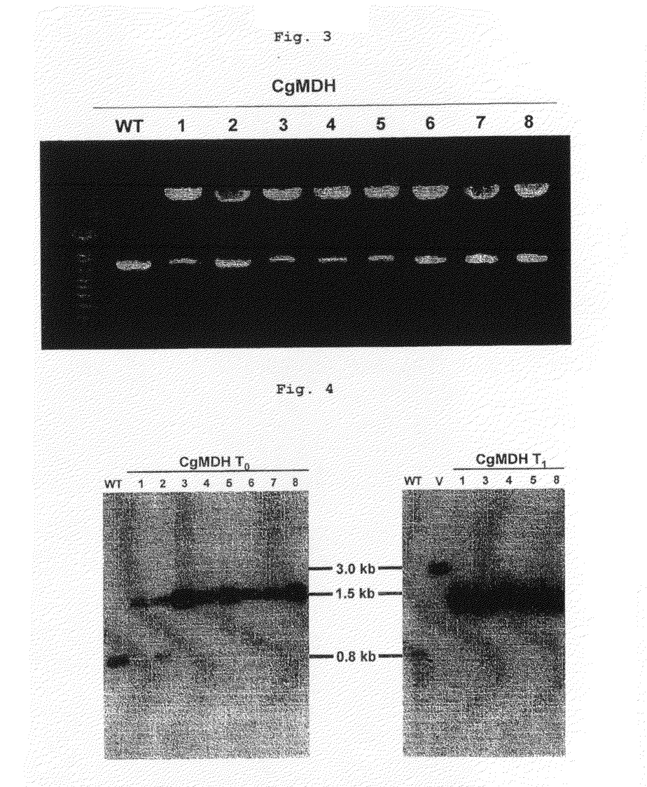

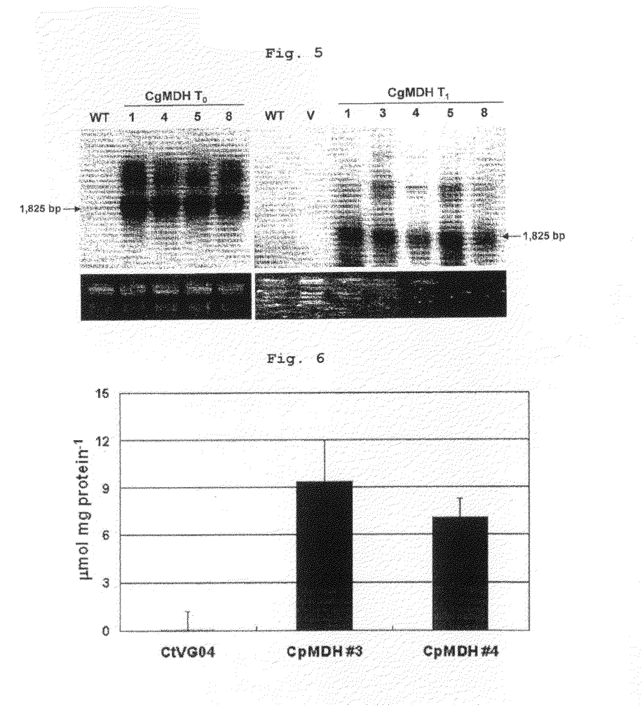

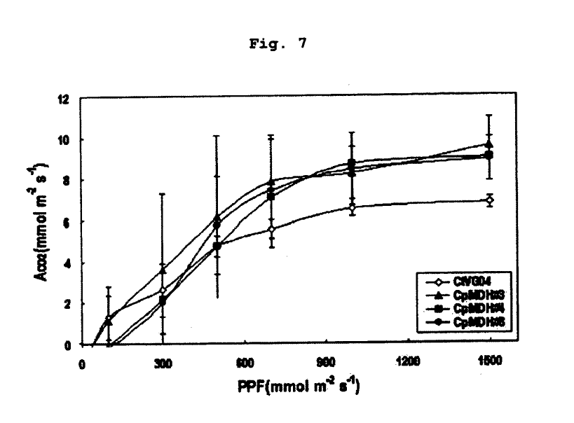

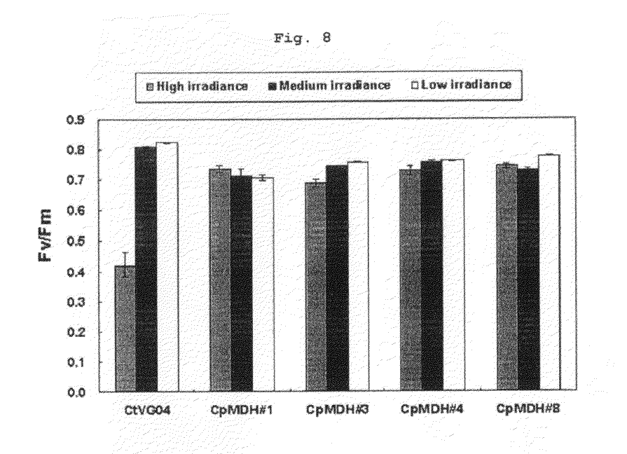

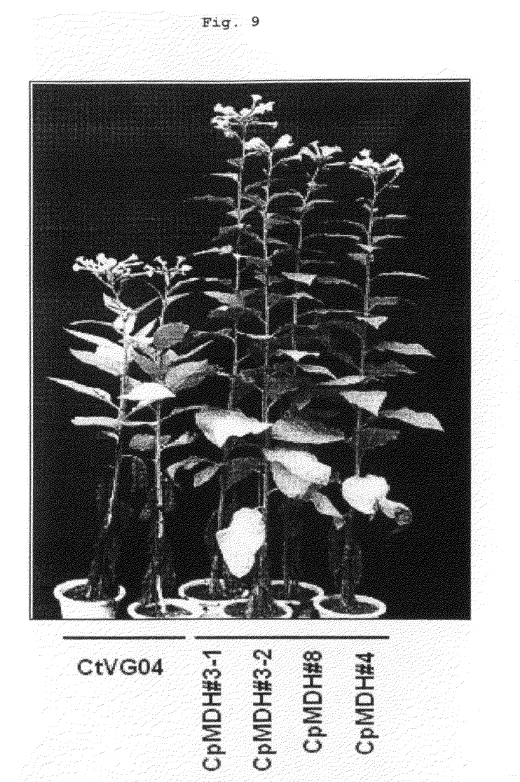

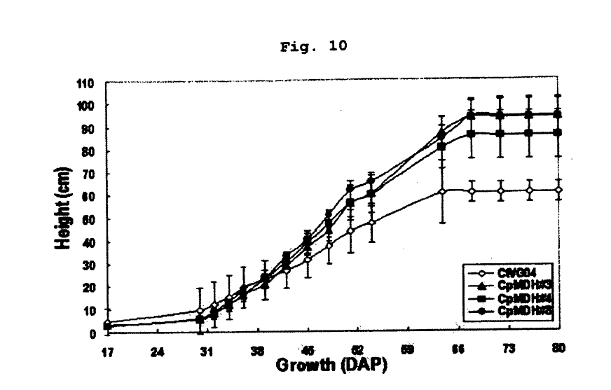

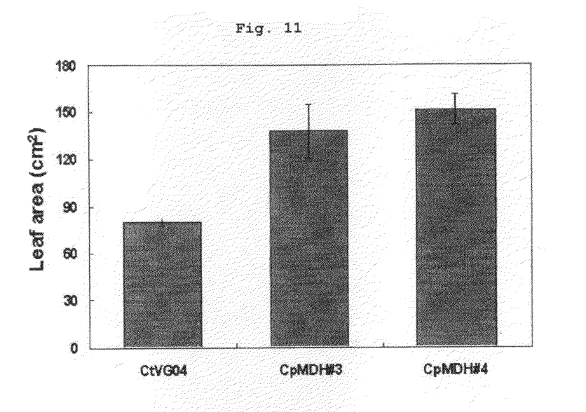

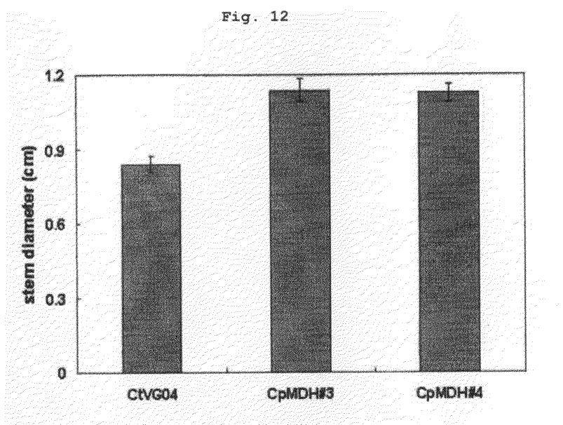

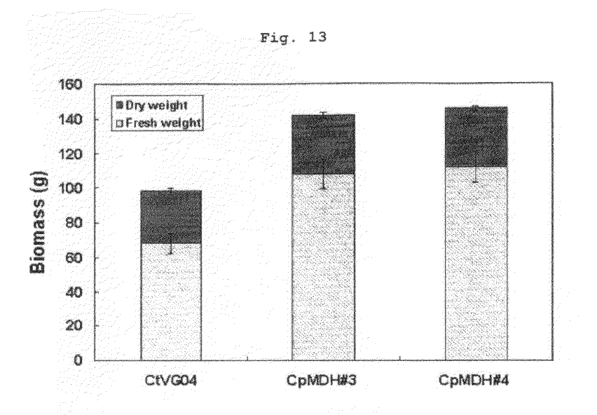

The plastid originated promoter is preferably clp promoter originated from rice (Oryza sativa), but not always limited thereto. The terminator herein is preferably rrnB1/B2 terminator originated from Escherichia coli pHCE19 vector, but not always limited thereto. The vector for plastid transformation herein additionally contains a selection gene which is preferably aadA or gfp, but not always limited thereto, and the vector is preferably the recombinant vector containing trnl gene sequence, trnA gene sequence, aadA gene sequence, gfp gene sequence and rbs sequence (see FIG. 1).

The present invention further provides a method for enhancing photosynthesis and biomass, which includes the step of cultivating the transgenic plant in outdoors or greenhouse under high luminosity condition.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Construction of Basic Vector for Plastid Transformation

The present inventors modified CtV2 vector for chloroplast transformation (Guda G, et al., Plant Cell Rep 19:257-26, 2000) provided by professor Daniell's lab, USA, and then constructed the typical vector CtVG04 for chloroplast transformation containing GFP by the same manner as used for the construction of pTlG vector (Jeong S W, et al., Plant Cell Rep, 22:747-751, 2004) (FIG. 1b). PCR amplification was performed using chromosome DNA of Oryza sativa as a template with rclpP5 primer (SEQ. ID. NO: 1) and rclpP3 primer (SEQ. ID. NO: 2), followed by cloning into pCR2.1-TOPO vector (Invitrogen, USA). Particularly, PCR was performed with exTaq enzyme (TaKaRa, Japan) by using 10-100 ng of genomic DNA of Oryza sativa as a template as follows; predenaturation at 94° C. for 5 minutes, denaturation at 94° C. for 1 minute, annealing at 55° C. for 1 minute, polymerization at 72° C. for 1 minute, 30 cycles from denaturation to polymerization, and final extension at 72° C. for 10 minutes. 800 bp rrnB1/B2 terminator (SEQ. ID. NO: 3) was separated from the commercial Escherichia coli expression vector pHCE19 (TaKaRa, Japan) by using PstI/HincII and used in this invention. The nucleotide sequence of Oryza sativa clp promoter was confirmed by sequencing (SEQ. ID. NO: 4).

To construct pTIA vector, PCR was performed with 10-100 ng of tobacco genomic DNA using I-L1 primer (SEQ. ID. NO: 5) and I-R2 primer (SEQ. ID. NO: 6) to amplify 1.95 kb trnI-trnA boarder DNA fragment. PCR was performed with exTaq enzyme (TaKaRa, Japan) as follows; predenaturation at 94° C. for 5 minutes, denaturation at 94° C. for 1 minute, annealing at 55° C. for 1 minute, polymerization at 72° C. for 2 minutes, 30 cycles from denaturation to polymerization, and final extension at 72° C. for 10 minutes. PCR product was digested with XbaI/KpnI and the ends were blunted by using Klenow enzyme, which was inserted into PvuII site of pUC18 vector (Fermentase, USA), resulting in the basic vector pTIA (FIG. 5c).

To construct pRclPADGHT vector (FIG. 5d), aadA-GFP DNA fragment separated from pTIG vector was introduced into SalI/PstI site of pBluescript KSII vector (Stratagene, USA), which was then digested with BamHI/SmaI. The rrnB1/B2 terminator DNA fragment was introduced into the end blunted by Klenow enzyme. Then, rclp promoter was inserted into XhoI/SalI site to construct pRclPADGHT vector.

To construct pRclPGAH vector (FIG. 5e), PCR was performed using pRclPADGHT vector as a template with aadA-PstI 5′ primer (SEQ. ID. NO: 7) and aadA-HindIII 3′ primer (SEQ. ID. NO: 8), followed by cloning into pCR2.1 TOPO TA vector (Invitrogen, USA) to construct pCR2.1 aadA(H/P) vector. PCR was performed with exTaq enzyme (TaKaRa, Japan) as follows; predenaturation at 94° C. for 5 minutes, denaturation at 94° C. for 30 seconds, annealing at 50° C. for 30 seconds, polymerization at 72° C. for 30 seconds, 27 cycles from denaturation to polymerization, and final extension at 72° C. for 5 minutes. DNA fragment obtained from pCR2.1 aadA(H/P) vector by digesting with PstI/HindIII was introduced into pRclPADGHT predigested with PstI/HindIII to construct double aadA vector. In the meantime, rclp promoter was amplified by PCR using pRclPADGHT vector as a template with rclp-XhoI primer (SEQ. ID. NO: 9) and rclp-MCS 3′ primer (SEQ. ID. NO: 10) containing SalI, XbaI, EcoRV and SacI sites in that order, followed by cloning into pCR2.1 TOPO TA vector (Invitrogen, USA) to construct pCR2.1 Rclp-MCS. PCR was performed by the same conditions as provided for the construction of pCR2.1 aadA(H/P). aadA vector was digested with XhoI/SacI to cut off the original rclp promoter and aadA gene, and instead rclp-MSC DNA fragment obtained by digesting Rclp-MCS with XhoI/SacI was inserted into the region, resulting in the construction of pRclPGAH.

### Example 2

### Construction of Vector for Plastid Transformation Containing MDH

The basic vector shown in FIG. 1 was used as a vector for plastid transformation containing MDH. First, PCR was performed using chromosome DNA of Corynebacterium glutamicum as a template with MDH 5′ primer (SEQ. ID. NO: 11) and MDH 3′ primer (SEQ. ID. NO: 12), followed by cloning into pGEM T-easy vector (Promega, USA). The constructed vector was named “TvecMDH”. PCR was performed with exTaq enzyme (TaKaRa, Japan) as follows; predenaturation at 94° C. for 5 minutes, denaturation at 94° C. for 30 seconds, annealing at 55° C. for 30 seconds, polymerization at 72° C. for 1 minute, 27 cycles from denaturation to polymerization, and final extension at 72° C. for 5 minutes. TvecMDN vector was digested with SalI/HindIII to cut MDH DNA, which was inserted into SalI/HindIII site of pRclPADGHT vector to construct RclpMDH vector. The RclpMDH vector was digested with XhoI/EcoRI and the ends were blunted by treating Klenow enzyme. The vector was then inserted into CtVG04 vector predigested with PstI (FIG. 1b) to construct the vector for plastid transformation (FIG. 2A). The vector was designed to have Corynebacterium glutamicum MDH gene in front of selection markers if necessary (FIG. 2B). The vectors shown in FIG. 2A and FIG. 2B have monocistronic structure in which MDH gene is transcribed by a single promoter. A vector designed to have polycistronic structure in which Corynebacterium glutamicum MDH gene is transcribed by clp promoter together with aadA and gfp selection genes was also constructed (FIGS. 2C, D, E and F). P1 is the probe used for Southern blotting and P2 is the probe used for Northern blotting. To perform Southern blotting, chromosome was digested with BglII [symbol] BamHI and the sizes of bands presented by P1 probe were indicated in schematic diagram of each vector.

### Example 3

### Construction of Plastid Transformed Tobacco Plant by Using the Vector for Plastid Transformation Containing MDH

Plastid transformation was performed by using the vector constructed in Example 2 and the vector CtVG04 constructed in Example 1 was used for the control group.

After germinating wild type tobacco (Nicotiana tabacum, Samsun) seeds in a chamber for 8 weeks, leaves were separated from the young plant and explanted in MS medium supplemented with 1 mg/l BAP and 0.1 mg/l NAA for further use in plastid transformation.

Gold particles (0.6 μm in diameter) were coated with the vectors for plastid transformation constructed in Examples 1 and 2 by using CaCl₂ and spermidine, followed by transformation using PDH-1000/He gene delivery system (BioRad, USA) under the conditions of 100 psi acceleration power, 9 cm target distance and 28 in/Hg vacuum.

The tobacco plant leaves treated above were cultured for 2 days at 25° C. under dark condition with 2,000 lux of light. The leaves were cut into 2-5 mm sections, which were cultured in MS medium supplemented with 1 mg/l BAP, 0.1 mg/l NAA and 500 mg/l spectinomycin for 6-7 weeks to induce resistant young shoot. The young shoot induced in the spectinomycin resistant medium was cut into 3 mm×3 mm pieces, followed by redifferentiation in the same selection medium to induce resistant young shoot again. The repeat of the redifferentiation in the selection medium resulted in the increase of homoplasmy of the introduced gene.

To confirm whether the redifferentiated plant (T₀) induced in the antibiotic containing medium was the transformant having the target gene or not, DNA was extracted from each induced plant, followed by PCR using vector specific trnI F3 primer (SEQ. ID. NO: 13) and trnA R1 primer (SEQ. ID. NO: 14). As a result, the target gene was successfully delivered into the transgenic plant. PCR was performed as follows; predenaturation at 95° C. for 5 minutes, denaturation at 95° C. for 45 seconds, annealing at 55° C. for 45 seconds, polymerization at 72° C. for 5 minutes, 30 cycles from denaturation to polymerization, and final extension at 72° C. for 15 minutes. Approximately 800 bp sized band amplified from a part of trnI and trnA was detected in wild type, while both about 800 bp sized band amplified from a part of trnI and trnA and about 5.1 kb sized band resulted from the introduction of a foreign gene in between trnI and trnA were detected at the same time in MDH transgenic plant (FIG. 3).

### Example 4

### Southern Blotting with Plastid Transformed Tobacco Plant

Southern blotting was performed using trnA gene as a marker to investigate homoplasmy of chloroplast in the T₀ and T₁ transformants obtained in Example 3. For the Southern blotting, total chromosome DNA was extracted by using a DNA extraction kit (DNeasy Plant Mini Kit, QIAGENE, Germany). 4 μg of the extracted DNA was digested with BamHI and BglII, which proceeded to electrophoresis on 1% agarose gel, followed by blotting on a blotting membrane (Zeta-Probe GT blotting membrane: Bio-Rad, USA). To prepare a probe, 0.6 kb BamHI/BglII DNA fragment containing trnA was labeled with [α-³²P]dCTP. Prehybridization and hybridization were performed using 0.25 M sodium phosphate buffer (pH 7.2) containing 7% (w/v) SDS at 65° C. for overnight. Upon completion of the reaction, the membrane was washed with 20 mM sodium phosphate buffer (pH 7.2) containing 5% (w/v) SDS at 65° C. for 30 minutes, which was then exposed for 3 hours.

As a result, 0.8 kb sized DNA band was detected in wild type, approximately 3.0 kb sized band was detected in the vector control group transformed with CtVG04 of Example 3, and 1.5 kb sized DNA band was detected in the MDH transformant. From the above results, it was confirmed that a target gene was introduced into the chloroplast genome of the redifferentiated transgenic plant and well-preserved during the transformation and this introduced foreign gene was delivered to the next generation as homoplasmy (FIG. 4).

### Example 5

### MDH Expression in Plastid Transformed Tobacco Plant

Northern blotting was performed to investigate whether the MDH gene introduced into the T₀ and T₁ transformants obtained in Example 3 was successfully transcribed. Total RNA for the Northern blotting was extracted using TRizol reagent (Invitrogen, USA). 2 μg of the extracted RNA proceeded to electrophoresis on 1.2% agarose gel supplemented with 5.1% (v/v) formaldehyde, followed by blotting on a blotting membrane (Zeta-Probe GT blotting membrane: Bio-Rad, USA). 1.044 bp MDH was amplified by PCR and the amplified product was labeled with [α-³²P]dCTP to prepare a probe. Prehybridization and hybridization were performed using 0.25 M sodium phosphate buffer (pH 7.2) containing 7% (w/v) SDS at 65° C. for overnight. Upon completion of the reaction, the membrane was washed with 20 mM sodium phosphate buffer (pH 7.2) containing 5% (w/v) SDS at 65° C. for 30 minutes, which was then exposed for 30 minutes.

As a result, no RNA bands were detected in wild type and the vector control plants, 1.8 kb sized RNA bands containing MDH and rrnB1/B2 terminator were detected in the redifferentiated MDH transformed T₀ and T₁ plants. In addition, various sized mRNAs linked by operon were also detected. The foreign gene introduced into plastid genome of the redifferentiated T₀ and T₁ transformants were well-preserved during the transcription, suggesting that the inserted gene can be translated as a protein functioning as an enzyme (FIG. 5).

### Example 6

### MDH Enzyme Activity of Plastid Transformed Tobacco Plant

NADP-dependent MDH enzyme activity of the T₀ and T₁ transformants obtained in Example 3 was measured with a spectrophotometer (Shimadzu UV-VIS Spectorophotometer, Japan). 6 hours after the sun rise, the 4^{th}-5^{th} fresh leaves from the top were taken and prepared in the size of 1.5 cm in diameter. Leaf tissues were separated using extraction buffer [100 mM HEPES-KOH (pH7.3), 10 mM MgCl₂, 2 mM K₂HPO₄, 1 mM EDTA, 10% glycerol, 10 mM β-mercaptoethanol, 10 mM NaF, 2 mM PMSF]. The leaves were homogenized fast by using a mortar at a low temperature, followed by centrifugation at 12000 rpm (4° C.) for 15 minutes. The supernatant was transferred in a new tube. When there were floating substances, centrifugation was performed one more time. The leaf extract which was standing at 4° C. for at least 30 minutes proceeded to Sephadex G-25 (1*3 cm) column (GE Healthcare Bio-Science AB, Sweden) to eliminate salts in order to be used for enzyme reaction. Enzyme reaction solution [50 mM HEPES-KOH (pH7.3), 5 mM MgCl₂, 0.2 mM NADPH, 2 U MDH, 5 mM EDTA, 2 mM OAA, 0.2 mM NADPH] was prepared. 1 Ml of NADP-dependent MDH enzyme reaction solution containing a proper amount of the leaf extract was treated at 30° C. and NADPH absorption rate was measured at 340 nm. The amount of protein included in the leaf extract was quantified by using protein assay kit (BIoRad, USA). As a result, the NADP-dependent MDH enzyme activity of the MDH transgenic plant was remarkably increased, compared with that of the vector control plant (FIG. 6).

### Example 7

### Photosynthesis Dependent CO₂ Absorption in Plastid Transformed Tobacco Plant

The T₀ and T₁ transformants prepared in Example 3 were cultivated in greenhouse. CO₂ absorption rate of a leaf without damage was measured with Ll-6400 photosynthesis system (Li-Cor, Inc., Lincoln, Nebr., USA). Red and blue LED lights were used as a light source and 6400 CO₂ mixture was used as CO₂ herein. To measure the CO2 absorption rate according to luminosity, a normal healthy leaf was fixed in a chamber provided with 400 μmol photons CO₂ mol⁻¹ air CO₂ at 25° C. and then CO₂ absorption rate was measured with changing light conditions (0, 100, 300, 500, 700, 1000 and 1500 μmol photons CO₂ mol⁻¹ s⁻¹).

As a result, CO₂ absorption rate of the MDH transgenic plant was approximately 1.41 fold increased at 1500 μmol photons compared with the control plant (FIG. 7).

### Example 8

### Photosystem II Efficiency According to the Increase of Luminosity in Plastid Transformed Tobacco Plant

Chlorophyll fluorescence is generally released from photosystem 2 (PSII). So, Fv/Fm (the ratio of fluorescence variation to maximum chlorophyll fluorescence) of the dark adapted leaf indicates the maximum value of quantum yield of photochemical reaction. That is, the value means the maximum, potential value to carry out photosynthesis in a leaf, which can also be an index of measuring the damage of photosystem 2. Normal healthy leaf maintained the Fv/Fm value as 0.8. The T₀ and T₁ transformants prepared in Example 3 were cultivated in greenhouse with different light conditions (high luminosity; 800˜1600 μmol photons/m²/sec, medium luminosity; 300˜800 μmol photons/m²/sec, low luminosity; 30˜300 μmol photons/m²/sec). The 5^{th} leaf from the top of the plant was taken and clipped in a potable chlorophyll fluorescence meter (Hansatech, King's Lynn, UK). After the dark treatment for 30 minutes, Fv/Fm value was measured at saturated luminosity. As a result, Fv/Fm values of plants growing under medium luminosity and low luminosity, regardless of the MDH transgenic plant or the control plant, were all maintained as 0.8. However, under high luminosity, the MDH transgenic plant maintained Fv/Fm value as 0.8 but the vector control plant exhibited reduced Fv/Fm value to 0.42. From the results, it was confirmed that photoinhibition by photosystem 2 was not induced in the MDH transgenic plant even under high luminosity condition (FIG. 8).

### Example 9

### Growth Rate and Biomass of Plastid Transformed Tobacco Plant

The T₀ and T₁ transformants prepared in Example 3 were cultivated in greenhouse and biomass increase was observed every 2-3 days until floral axis was developed. To evaluate biomass increase rate, growth rate, leaf area, stem diameter, live weight and dry weight of a plant were all considered. To measure the leaf area, the 13^{th} leaf from the bottom of a plant was selected and the leaf area was measured by using a soft ware. Stem diameter was measured 3 cm up from the bottom of a plant using a ruler (Manostat, 15-100-500). Roots, stems and leaves were separated from a plant and live weight of each was measured by a balance. They were dried in an 80° C. oven for 6 days, and dry weight of each was measured. Growth rate of the MDH transgenic plant was rapidly increased from the 38^{th} day after transplantation to the soil and 40-55% increased on around the 65^{th} day when floral axis was developed, compared with the vector control plant (FIGS. 9 and 10). Compared with the vector control plant, leaf area of the MDH transgenic plant was 88% increased, stem diameter was 35% increased, live weight was 63% increased and dry weight was 14% increased (FIGS. 11-13).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A transgenic plant prepared by inserting MDH (malate dehydrogenase) gene originated from a bacteria into the plastid genome of a target plant.

2. The transgenic plant according to claim 1, wherein the bacteria is selected from the group consisting of Corynebacterium glutamicum, Rhodococcus, Oceanobacillus, Aspergillus, Methanothermus, Chaetomium, Methanopyrus, Bacillus, Methanocaldococcus, Magnaporthe, Phaeosphaeria, Gibberella, Desulfitobacterium, coccidioides, Thermus, Candidatus, Pyrococcus, Solibacter, Aurantimonas, Syntrophus, Enterococcus, Methanosphaera, Anopheles, Entamoeba, Yersinia, Mesorhizobium, Tribolium, Salmonella, Aurantimonas, Amycolatopsis, Escherichia coli, Apis, Burkhoderia, Bordetella, Pseudomonas and Aedes.

3. The transgenic plant according to claim 1, wherein the bacteria is Corynebacterium glutamicum.

4. The transgenic plant according to claim 1, wherein the target plant is C3 plant.

5. The transgenic plant according to claim 4, wherein the C3 plant is selected from the group consisting of tobacco; cereals including rice; beans including mung bean, kidney bean and pea; starch-storing plants including potato, cassava and sweet potato; oil-storing plants including soybean, rape, sunflower and cotton plant; vegetables including tomato, eggplant, carrot, hot pepper, Chinese cabbage, radish, water melon, cucumber, melon, crown daisy, spinach, cabbage and strawberry; garden plants including chrysanthemum, rose, carnation and petunia; and Arabidopsis.

6. A method for preparing a transgenic plant with the insertion of MDH gene originated from a bacteria in its plastid genome, comprising the following steps:
1) constructing a vector for MDH plastid transformation by inserting MDH (malate dehydrogenase) gene sequence which is active in plastid into a vector for plastid transformation;
2) preparing a transformant by introducing the vector for MDH plastid transformation into C3 plant or C3 plant culture cell;
3) culturing the transformant of step 2); and
4) re-differentiating the cultured transformant of step 3) by tissue culture.

7. The method for preparing a transgenic plant according to claim 6, wherein the bacteria of step 1) is selected from the group consisting of Corynebacterium glutamicum, Rhodococcus, Oceanobacillus, Aspergillus, Methanothermus, Chaetomium, Methanopyrus, Bacillus, Methanocaldococcus, Magnaporthe, Phaeosphaeria, Gibberella, Desulfitobacterium, coccidioides, Thermus, Candidatus, Pyrococcus, Solibacter, Aurantimonas, Syntrophus, Enterococcus, Methanosphaera, Anopheles, Entamoeba, Yersinia, Mesorhizobium, Tribolium, Salmonella, Aurantimonas, Amycolatopsis, Escherichia coli, Apis, Burkhoderia, Bordetella, Pseudomonas and Aedes.

8. The method for preparing a transgenic plant according to claim 6, wherein the bacteria of step 1) is Corynebacterium glutamicum.

9. The method for preparing a transgenic plant according to claim 8, wherein the MDH gene is represented by SEQ. ID.

10. The method for preparing a transgenic plant according to claim 6, wherein the expression vector for plastid transformation of step 1) contains the gene construct composed of a plant plastid originated promoter that has low homology with the polynucleotide of the genome of a target plant plastid, rbs sequence, and a terminator that has low homology with the polynucleotide of the genome of a target plant plastid in that order.

11. The method for preparing a transgenic plant according to claim 10, wherein the target plant is C3 plant.

12. The method for preparing a transgenic plant according to claim 11, wherein the C3 plant is selected from the group consisting of tobacco; cereals including rice; beans including mung bean, kidney bean and pea; starch-storing plants including potato, cassava and sweet potato; oil-storing plants including soybean, rape, sunflower and cotton plant; vegetables including tomato, eggplant, carrot, hot pepper, Chinese cabbage, radish, water melon, cucumber, melon, crown daisy, spinach, cabbage and strawberry; garden plants including chrysanthemum, rose, carnation and petunia; and Arabidopsis.

13. The method for preparing a transgenic plant according to claim 10, wherein the plastid originated promoter is clp promoter originated from Oryza sativa.

14. The method for preparing a transgenic plant according to claim 10, wherein the terminator is rrnB1/B2 terminator originated from Escherichia coli pHCE19 vector.

15. The method for preparing a transgenic plant according to claim 10, wherein the vector for plastid transformation additionally includes a selection gene.

16. The method for preparing a transgenic plant according to claim 15, wherein the selection gene is aadA or gfp.

17. A method for enhancing photosynthesis and biomass of a plant, which includes the step of cultivating the transgenic plant of claim 1 in outdoors or greenhouse under the high luminosity condition.


SEQUENCE LISTING (16 sequences)

SEQ ID NO: 1 - DNA, 26 bp, Artificial Sequence
  Synthetic construct  rcplP5 primer
gcgctgcagcttatttggaaaaaacg

SEQ ID NO: 2 - DNA, 33 bp, Artificial Sequence
  Synthetic construct  rcplP3 primer
gcgtcgactccctcctaagtaagaaaactacac

SEQ ID NO: 3 - DNA, 781 bp, Artificial Sequence
  Synthetic construct  rrnB1/B2 terminator
ctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgt

SEQ ID NO: 4 - DNA, 261 bp, Artificial Sequence
  Synthetic construct  clp promoter
gcttatttggaaaaaacgaagaatagatccctatctctttttgtttagtattcgaatcaccattcttttttctttattcaatctgtcttatcctacttatatgtataatctttcaatctatgtattatttcaatctacgtacttaatagaatctatagtattcatatagaataagaaaaaaacgtgaaaacaataaactgcggattctttctttctcttccattcttacgtttccatattaaagtgtagttttcttactta

SEQ ID NO: 5 - DNA, 25 bp, Artificial Sequence
  Synthetic construct  I-L1 primer
gctctagattctcgacggtgaagta

SEQ ID NO: 6 - DNA, 25 bp, Artificial Sequence
  Synthetic construct  I-R2 primer
ggggtaccttaaggctatgccatcc

SEQ ID NO: 7 - DNA, 36 bp, Artificial Sequence
  Synthetic construct  aadA-PstI 5′
ctgcagaggaggtataacatgagggaagcggtgatc

SEQ ID NO: 8 - DNA, 26 bp, Artificial Sequence
  Synthetic construct  aadA-HindIII 3′
aagcttttatttgccgactaccttgg

SEQ ID NO: 9 - DNA, 25 bp, Artificial Sequence
  Synthetic construct  rclp-XhoI 5′
ctcgagcttatttggaaaaaacgaa

SEQ ID NO: 10 - DNA, 42 bp, Artificial Sequence
  Synthetic construct  rclp-MCS 3′
gagctcgatatctctagagtcgactaagtaagaaaactacac

SEQ ID NO: 11 - DNA, 34 bp, Artificial Sequence
  Synthetic construct  MDH 5′
gtcgacaggaggtatacaatgccagaagtcactg

SEQ ID NO: 12 - DNA, 28 bp, Artificial Sequence
  Synthetic construct  MDH 3′
aagctttcaacggtgagttacaacatgg

SEQ ID NO: 13 - DNA, 28 bp, Artificial Sequence
  Synthetic construct  trnI F3
aagctttcaacggtgagttacaacatgg

SEQ ID NO: 14 - DNA, 20 bp, Artificial Sequence
  Synthetic construct  trnA R1
gttcttgacagcccatcttt

SEQ ID NO: 15 - protein, 347 aa, Corynebacterium glutamicum
MPEVTVNAQQLTVLCTDILTKTGVPAADAHLVGDSLVQADLWGHPSHGVLRLPWYVRRLHSGAMTTHAHVEVLNDLGAVLALDGHNGIGQVLADHARKEAVTRAMMFGIGAVSVRNSNHFGTAMYYTRKAAAQGCVSILTTNASPAMAPWGGREKRIGTNPWSIAAPFGETATVVDIANTAVARGKIYHARQTNMPIPETWAITSEGAPTTDPAEAINGVVLPMAGHKGYAISFMMDVLSGVLTGSQHSTKVHGPYDPTPPGGAGHLFIALDVAAFRDPQDFDDALSDLVGEVKSTPKAQNTEEIFYPGESEDRAHRKNSAHGISLPEKTWMELQELAIENHVVTHR

SEQ ID NO: 16 - DNA, 1044 bp, Corynebacterium glutamicum
atgccagaagtcactgtcaacgcccaacaactcactgttctctgcacagacatcctcaccaaaactggagtacctgcagcagacgcccatcttgtcggtgatagtttggtgcaggctgatctttggggtcacccctcccacggtgtgcttcgactgccttggtatgtgcgcagactccacagtggcgcgatgactacacatgcacacgtggaggttctcaatgatttgggtgccgtgttggcgttggatggacacaatggaatcggccaagttttagctgatcatgctcgtaaagaagcagtgactagggcaatgatgttcggcatcggtgcggtgtcggtgcgcaactccaatcattttggaactgccatgtactacacccggaaagcggcagcgcaaggatgtgtttccattctcaccaccaatgcatctccggcgatggcgccctggggtggcagagaaaaacggatcggtaccaacccatggtctattgcggcaccttttggagaaacggctacggtagtcgatatagccaatactgcggttgcgcgcggaaagatctaccacgcacgacagacaaacatgcccattcctgagacttgggcgatcacgagtgagggcgcacccaccacggatcctgctgaggcaatcaacggtgtcgtccttcccatggctggtcacaaaggttatgcgattagcttcatgatggatgtgctttctggagttctcactggttcccagcacagcaccaaggtacatggtccgtatgatcccactcccccaggtggagctggccacttgttcatcgcgttggatgttgcagcgtttcgcgatccacaagatttcgatgacgcactcagcgatctggttggggaagttaaatccactccgaaagcacaaaacaccgaggagattttctaccccggtgaatcggaagaccgtgcgcatcggaaaaactctgcgcacggtatttcattgcctgaaaaaacgtggatggaactgcaagaactggcaatcgagaaccatgttgtaactcaccgttga